(19) United States
(12) Patent Application Publication
(10) Pub. No.: US 2016/0346320 A1
(43) Pub. Date: Dec. 1, 2016

(54) SUBSTANCE FOR INHIBITING TISSUE CALCIFICATION, TISSUE FIBROSATION AND AGE-RELATED DISEASES

(71) Applicant: Fresenius Kabi Deutschland GmbH, Bad Homburg (DE)

(72) Inventors: Florian Lang, Tuebingen (DE); Christina Leibrock, Tuebingen (DE); Ioana Alesutan, Tuebingen (DE)

(21) Appl. No.: 15/025,383

(22) PCT Filed: Sep. 24, 2014
(86) PCT No.: PCT/EP2014/070333

§ 371 Date: Jul. 11, 2016

(30) Foreign Application Priority Data
Sep. 26, 2013 (DE) 10 2013 110 608.0

(51) Int. Cl. A61K 33/02; A23L 33/16; A23L 33/10; A61K 31/433; A61K 31/4706

(52) U.S. Cl. CPC A61K 33/02; A61K 31/433; A61K 31/4706; A23L 33/10; A23L 33/16; A23V 2002/00

(57) ABSTRACT

A substance for reducing tissue calcification and tissue fibrosation, and for delaying the onset of age-related diseases of a living being, and associated methods.

The present invention relates to the use of a substance for inhibiting tissue calcification and tissue fibrosation, and for delaying the onset of age-related diseases and methods associated therewith.

The aging process of a living being is typically characterized by the increasing onset of diseases and organ disfunctions. Such so-called age-related diseases or aging syndromes ultimately result in the death of the being. Tissue calcification and tissue fibrosation play a decisive role in the aging process.

The tissue calcification plays a decisive role in particular in the accelerated aging of patients with renal failure. The decline of functioning organ tissue when replaced by connective tissue (fibrosation) plays a central role in renal failure, cirrhosis of the liver, Crohn's disease, fibrotic pancreatitis, pulmonary fibrosis, heart failure and scarring. Furthermore, tissue fibrosation leads to an impairment of the effectiveness of peritoneal dialysis.

Tissue calcification and fibrosation are both stimulated by the “transforming growth factor” TGFβ1, which also contributes to the development of Alzheimer's disease. An activation of the alkaline phosphatase and the increased expression of the transcription factor Runx2 also contribute to the signal transduction of tissue calcification.

Excessive tissue calcification, early onset of age-related diseases and shortened lifespans has been observed in mice with a klotho deficiency (kLot^{hm}). With an inhibition of the tissue calcification and extension of the lifetime after an agent has been administered to the mouse it can therefore be concluded that this agent delays tissue calcification and aging.

There are numerous references in the prior art to reputed substances for reducing tissue calcification and tissue fibrosation, and for delaying the onset of age-related diseases. In most cases, however, there is no scientific evidence for the effectiveness of these substances.

Based on this, the invention addresses the object of finding a substance for reducing tissue calcification and organ fibrosation, as well as age-related diseases in a living being.

This object is achieved by providing ammonium sulfate, ammonium chloride, the carbonic anhydrase inhibitor acetazolamide, chloroquine, ammonium nitrate, ammonium citrate, or ammonium lactate.

This discovery on the part of the inventor is surprising.

Ammonium sulfate is the salt from ammonia and sulfuric acid. In food technology, ammonium sulfate is used as an additive for regulating acidity, and is generally regarded as safe by the U.S. Food and Drug Administration (generally regarded as safe: [GRAS]). In the European Union it has the number E517.

Ammonium citrate is the salt from ammonia and citric acid, and is approved under the number E380.

Ammonium lactate is the salt from ammonia and lactic acid, and is listed in the European Union under the number E328 as an acidity regulator.

Ammonium nitrate is used as fertilizer and in explosives.

Ammonium chloride having the molecular formula NH₄Cl, also referred to as ammonium muriate, ammonia salt, or sal ammoniac, and having the CAS-No. 12125/02/9, is the ammonium salt of hydrochloric acid. It is a colorless, crystalline solid. Ammonium chloride is used in food technology as an additive, and has the number E 510. In medicine, ammonium chloride is used as an expectorant, i.e. as mucus expectorants.

Chloroquine [(RS)—N′-(7-chloroquinoline-4-yl)-N,N-diethyl-pentane-1,4-diamine] alkalizes lysosomes and is used against malaria, for immunosuppression, for treating viral diseases, and to combat tumors.

The carbonic anhydrase inhibitor acetazolamide inhibits the enzymatic conversion of bicarbonate to carbon dioxide, and can therefore act on the local pH. It is used as a diuretic.

It is known that an acidosis, such as can be triggered by ammonium chloride (NH₄Cl), can inhibit tissue calcification. It is furthermore known that acetazolamide can lower the phosphate concentration, resulting in a reduction in tissue calcification. In the experiments on the klotho^{hm} mouse, the tissue calcification is inhibited by ammonium chloride, but without increasing the acidosis, and by acetazolamide, without lowering the plasma phosphate concentration (see below).

The use of ammonium sulfate, ammonium citrate, ammonium lactate, ammonium nitrate, ammonium chloride (NH₄Cl), chloroquine or acetazolamide for inhibiting signal transduction, which leads to tissue calcification and tissue fibrosation, and delays the onset of age-related diseases, is not described in the prior art.

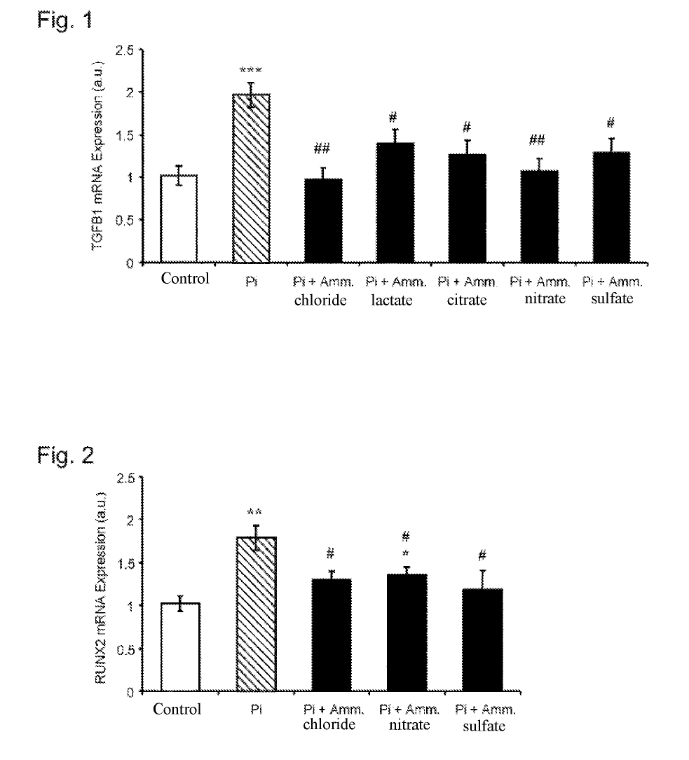

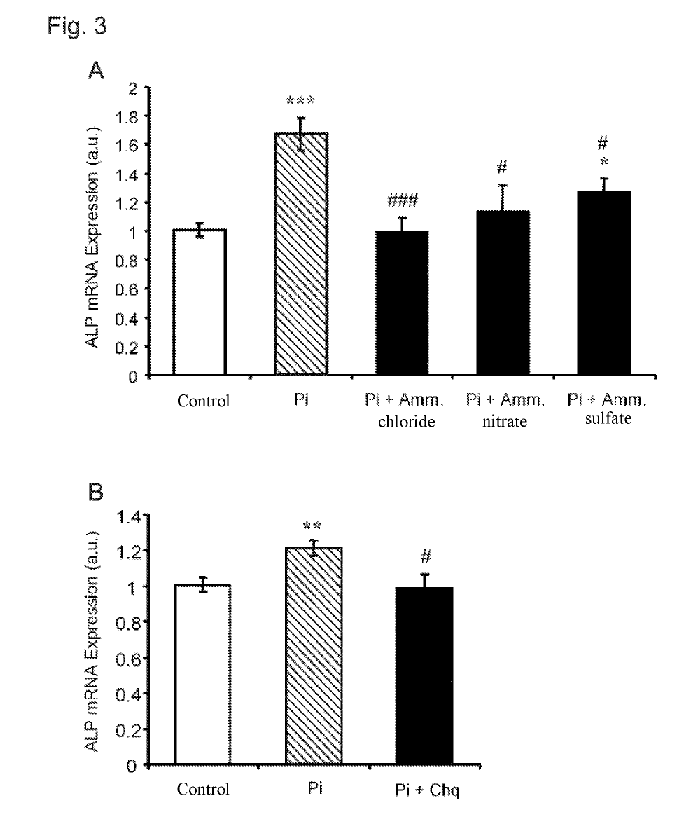

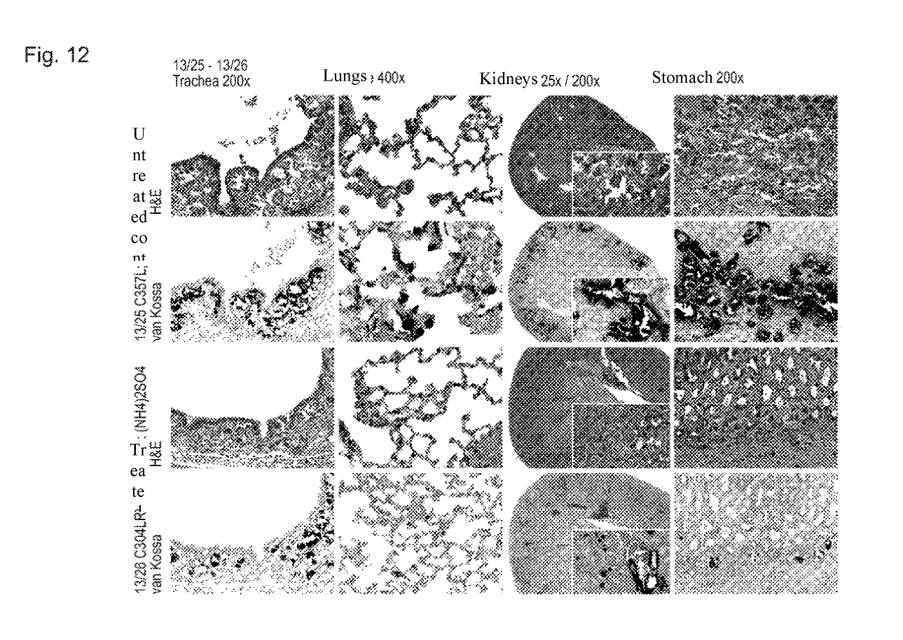

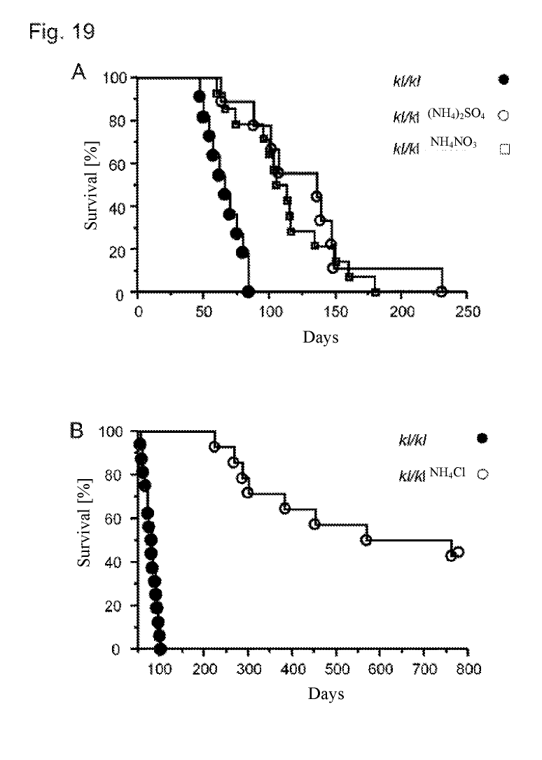

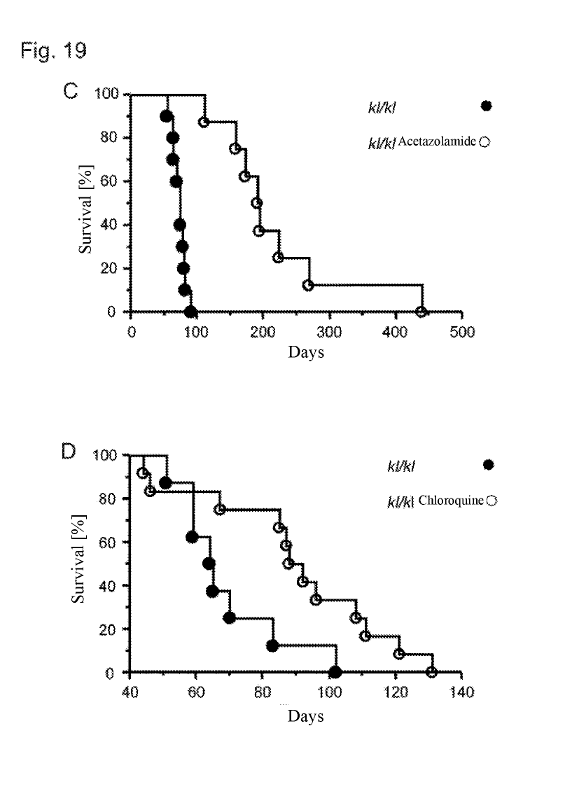

The inventor was able to prove, on an established cell model, that ammonium sulfate, ammonium citrate, ammonium lactate, ammonium nitrate and ammonium chloride (NH₄Cl) inhibit the formation of TGFβ1, a key molecule in the regulation of tissue calcification and tissue fibrosation (FIG. 1). Furthermore, the inventor was able to prove that ammonium sulfate, ammonium nitrate and ammonium chloride inhibit the expression of the transcription factor Runx2 (FIG. 2), and that ammonium sulfate, ammonium nitrate, ammonium chloride and chloroquine reduce the expression of alkaline phosphatases (FIG. 3), both of which are known stimulators of tissue calcification. Lastly, the inventor was able to show, in an established animal model, that the administration of ammonium chloride and acetazolamide leads to a clear extension of the lifetime (FIG. 19) and reduces or prevents tissue and vessel calcification (FIGS. 12-18).

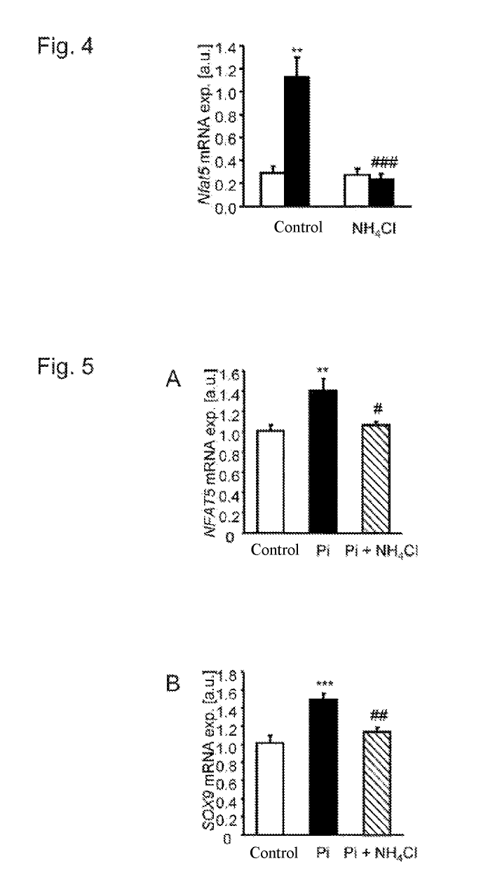

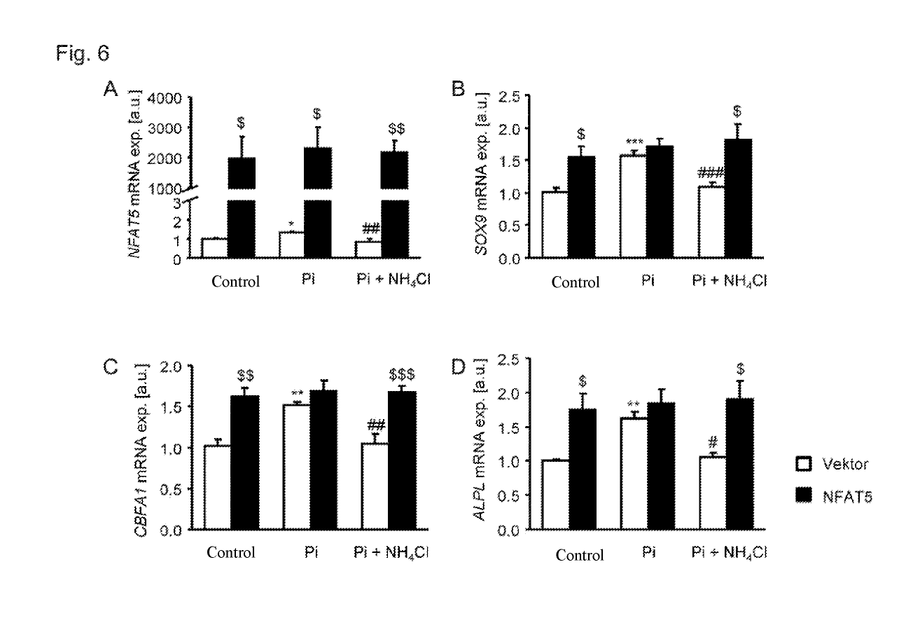

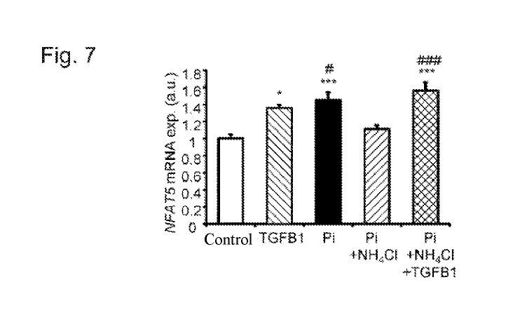

More extensive tests provided insight into the participating cellular mechanisms: it could be demonstrated that aortas of klotho-deficient animals exhibit a massive expression of the transcription factor NFAT5 (FIG. 4). It was possible to increase the expression of the transcription factor in cells through an increased extracellular phosphate concentration (FIG. 5A). At the same time, the expression of SOX9 increased, likewise a protein contributing to osteogenic signal transduction (FIG. 5B). Transfection of the cells with NFAT5 increases the expression of SOX9, CBFA1/RUNX2 and ALP, independently of phosphate, and prevents the effect of NH₄Cl on the expression of SOX9, CBFA1/RUNX2 and ALP (FIG. 6). Treatment of the cells with tumor growth factor TGFβ increases in turn, independently of phosphate, the expression of NFAT5 and prevents the effect of NH₄Cl on the expression of NFAT5 (FIG. 7).

In accordance with the invention, “use” is to be understood to mean that at least one of the specified substances induces the claimed effect. In accordance with the invention thereby, the use of the specified substances can occur in the framework of monotherapies, in which ammonium sulfate, ammonium citrate, ammonium lactate, ammonium nitrate, ammonium chloride (NH₄Cl), chloroquine and acetazolamide are used as the active substance, or the sole active substance, respectively. Combination therapies may also be implemented, however, in which two or more of these active substances are deployed simultaneously.

The fundamental objective of the invention is fully achieved herewith.

According to a preferred further development of the invention, age-related diseases are selected from the group composed of: arterial sclerosis, pulmonary emphysema, atrophoderma, myasthenia, acquired immune deficiency syndrome, infertility, kyphosis, disruption of the CaPO₄ metabolism, osteoporosis, low immunity (thymus degeneration), and neurodegeneration.

According to a preferred further development of the invention, the tissue fibrosation is based on a disease selected from the group composed of cirrhosis of the liver, Crohn's disease, fibrotic pancreatitis, pulmonary fibrosis, heart failure, scarring, fibrosation in peritoneal dialysis, Alzheimer's disease.

The measure has the advantage that substances are provided with which important fibrotic diseases can be treated prophylactically and therapeutically.

Ammonium sulfate, ammonium citrate, ammonium lactate, ammonium nitrate, ammonium chloride (NH₄Cl), chloroquine and acetazolamide can be substances in a pharmaceutical compound, which is preferably designed for an oral, rectal, parenteral, intraperitoneal, local or transdermal application. Furthermore, the pharmaceutical composition can preferably be designed as a powder, tablet, juice, drops, dialysis fluid, capsule, suppository, solution, injection solution, aerosol, ointment, rinse, patch, pellet, lozenge, or modified release dosage form.

The absolute quantity of ammonium sulfate, ammonium citrate, ammonium lactate, ammonium nitrate, ammonium chloride (NH₄Cl), chloroquine and acetazolamide in a dosage unit of the pharmaceutical composition is determined by the person skilled in the art on a case-by-case basis. Compositions for administration to adult humans may provide for daily doses of ca. 25 g ammonium sulfate, 25 g ammonium citrate, 35 g ammonium lactate, 25 g ammonium nitrate, 20 g ammonium chloride (NH₄Cl), 900 mg chloroquine, and 800 mg acetazolamide. The person skilled in the art may, however, provide other absolute quantities deviating therefrom.

This measure has the advantage that the active substance is provided in an absolute quantity that achieves the desired effect.

The pharmaceutical composition can contain a pharmaceutically acceptable carrier, and possibly other additives, which are generally known in the prior art. They are described, by way of example, in the publication by Kibbe, A., Handbook of Pharmaceutical Excipients, third edition, American Pharmaceutical Association and Pharmaceutical Press 2000. Additives comprise any compound or composition that is advantageous for the intended use of the compound according to the invention, which include salts, binders, solvents, dispersants, and other substances typically used in conjunction with the formulation of pharmaceuticals.

Ammonium sulfate, ammonium citrate, ammonium lactate, ammonium nitrate, ammonium chloride (NH₄Cl), chloroquine and acetazolamide can/may be used according to the invention as additive(s) in a food product.

This measure takes advantage of the fact that, in part, the substances have already been put to use in food technology, and are distinguished by tolerability and substantial tastelessness. Any arbitrary food product may be considered according to the invention, in particular beverages, but also solid foods.

The preferred concentration of the active substance can be readily determined by means of methods known to the person skilled in the art, e.g. via titration experiments, in which different concentrations are deployed. The effective quantity can be established on an individual basis. In the case of a therapeutic application, the concentration is based on the concrete age-related disease that is to be treated, the course, the severity, the patient that is to be treated, in particular according to his immune response status, sex, age, disease history, etc. When used in beverages, the concentration can amount to ca. 25 g ammonium sulfate, 25 g ammonium citrate, 35 g ammonium lactate, 25 g ammonium nitrate, 20 g ammonium chloride (NH₄Cl), 800 mg chloroquine, or 800 mg acetazolamide. The person skilled in the art may, however, also make use of concentrations deviating therefrom.

This measure has the advantage that the active substance, or the additive, is already provided in such a concentration that it ensures the desired effect.

A further subject matter of the invention relates to a method for producing a pharmaceutical composition for reducing tissue calcification and tissue fibrosation, as well as for delaying the onset of age-related diseases, having the following steps:
1. Provision of an active substance, and
2. Formulating the active substance in a pharmaceutically acceptable carrier for containing the pharmaceutical composition,
   wherein the active substance is selected from the group composed of: ammonium sulfate, ammonium chloride (NH₄Cl), acetazolamide, chloroquine, ammonium nitrate, ammonium citrate and ammonium lactate.

Moreover, a further subject matter of the present invention relates to a method for producing a food product for reducing tissue calcification and tissue fibrosation, as well as for delaying the onset of age-related diseases, having the following steps:
1. Provision of an additive, and
2. Introduction of the additive into a foodstuff in order to obtain the food product,
   wherein the additive is selected from the group composed of: ammonium sulfate, ammonium chloride (NH₄Cl), acetazolamide, chloroquine, ammonium nitrate, ammonium citrate and ammonium lactate.

Lastly, a further subject matter of the present invention relates to a method for reducing tissue calcification and tissue fibrosation, as well as for delaying the onset of age-related diseases, which comprises the administration of a substance to the living being, wherein the substance is selected from the group composed of: ammonium sulfate, ammonium chloride (NH₄Cl), acetazolamide, chloroquine, ammonium nitrate, ammonium citrate and ammonium lactate.

The properties, features and advantages of the use according to the invention apply accordingly to the methods according to the invention. As such, the substances can be used individually as sole active substances or additives, or in combinations thereof.

It is to be understood that the features specified above and to be explained below can be used not only in the respective given combinations, but also in other combinations or in and of themselves, without abandoning the scope of the present invention.

The invention shall now be explained in greater detail based on exemplary embodiments from which further properties and advantages can be derived. The exemplary embodiments are purely illustrative, and do not limit the scope of the invention. Reference is made thereby to the attached drawings.

In the attached Figures:

FIG. 1 shows the expression of TGFβ1 mRNA in human aortic smooth muscle cells (HAoSMCs) at normal phosphate concentrations (white column) and after increasing the phosphate concentration by adding 2 mM β-glycerophosphate to stimulate osteogenic signal transduction in the absence (grey column) and presence (black columns) of different ammonium salts (0.5 mM each). ***(p<0.001) shows a significant difference to normal phosphate concentrations; #(p<0.05), ##(p<0.01) show a statistically significant difference to increased phosphate concentrations in the absence of ammonium salts (student's t-test, n=4).

FIG. 2 shows the expression of Runx2 mRNA in human aortic smooth muscle cells (HAoSMCs: human aortic smooth muscle cells) at normal phosphate concentrations (white column) and after increasing the phosphate concentration by adding 2 mM β-glycerophosphate to stimulate osteogenic signal induction in the absence (grey column) and presence (black columns) of different ammonium salts (0.5 mM each). **(p<0.01), shows a statistically significant difference to normal phosphate concentration; #(p<0.05), ##(p<0.01), show a statistically significant difference to increased phosphate concentration in the absence of ammonium salts (student's t-test, n=6).

FIG. 3 shows the expression of alkaline phosphatase in mRNA in human aortic smooth muscle cells (HAoSMCs: human aortic smooth muscle cells) at normal phosphate concentrations (white column) and after increasing the phosphate concentration by adding 2 mM β-glycerophosphate to stimulate osteogenic signal induction in the absence (grey column) and presence (black columns) of different ammonium salts (FIG. 3A) (0.5 mM each) as well as after adding chloroquine (100 μM) (FIG. 3B). *(p<0.05), **(p<0.01), ***(p<0.001) show a statistically significant difference to normal phosphate concentration; #(p<0.05), ##(p<0.01), ###(p<0.001) show a statistically significant difference to increased phosphate concentration in the absence of ammonium salts or chloroquine (student's t-test, n=8).

FIG. 4 shows the expression of NFAT5 (nuclear factor of activated T-cells 5) in aortas of klotho^{+/+} mice (light bars) and klotho^{hm} mice (dark bars), in each case without (control) and with NH₄Cl treatment. (n=10) **(p<0.01) shows a statistically significant difference to klotho^{+/+} mice; ###(p<0.001) shows a statistically significant difference to untreated klotho^{hm} mice (ANOVA).

FIG. 5 shows the expression of NFAT5 (A) and SOX9 (B) in human aortic smooth muscle cells (HAoSMCs: human aortic smooth muscle cells) at normal phosphate concentrations (white column) and after increasing the phosphate concentration by adding 2 mM β-glycerophosphate to stimulate osteogenic signal induction in the absence (grey column) and presence (black columns) of ammonium chloride (500 μM). (n=6-8) **(p<0.01) shows a statistically significant difference to normal phosphate concentration; #(p<0.05) shows a statistically significant difference to increased phosphate concentration in the absence of ammonium chloride (ANOVA).

FIG. 6 shows the expression of NFAT5 (A), SOX9 (B), CBFA1/RUNX2 (C) and ALPL (D) in human aortic smooth muscle cells (HAoSMCs: human aortic smooth muscle cells) at normal phosphate concentrations (control) and after increasing the phosphate concentration by adding 2 mM β-glycerophosphate to stimulate osteogenic signal induction in the absence (Pi) and presence (Pi+NH₄Cl) of ammonium chloride (500 μM) after the control transfection (white columns) and transfection with a construct for encoding NFAT5 (black columns). (n=6) **(p<0.05), **(p<0.01), ***(p<0.001) show a statistically significant difference to normal phosphate concentration with control transfection (ANOVA); #(p<0.05), ##(p<0.01), ###(p<0.001) show a statistically significant difference to increased phosphate concentration in the absence of ammonium chloride with control transfection (ANOVA). $(p<0.05), $$(p<0.01), $$$(<0.001) show a statistically significant difference to control transfected HAoSMCs (t-test).

FIG. 7 shows the expression of NFAT5 in human aortic smooth muscle cells (HAoSMCs: human aortic smooth muscle cells) at normal phosphate concentrations (control), in the presence of TGF-beta (10 ng/ml; TGFβ1), after increasing the phosphate concentration by adding 2 mM β-glycerophosphate to stimulate osteogenic signal induction in the absence (Pi) and presence (Pi+NH₄Cl) of ammonium chloride (500 μM), as well as in the presence of an increased phosphate concentration, together with TGF-beta and ammonium chloride (Pi+NH₄Cl+TGFβ1). (n=6) *(p<0.05), ***(p<0.001) show a statistically significant difference to the normal phosphate concentration; #(p<0.05), ###(p<0.001) show a statistically significant difference to the increased phosphate concentration in the absence of ammonium chloride (ANOVA).

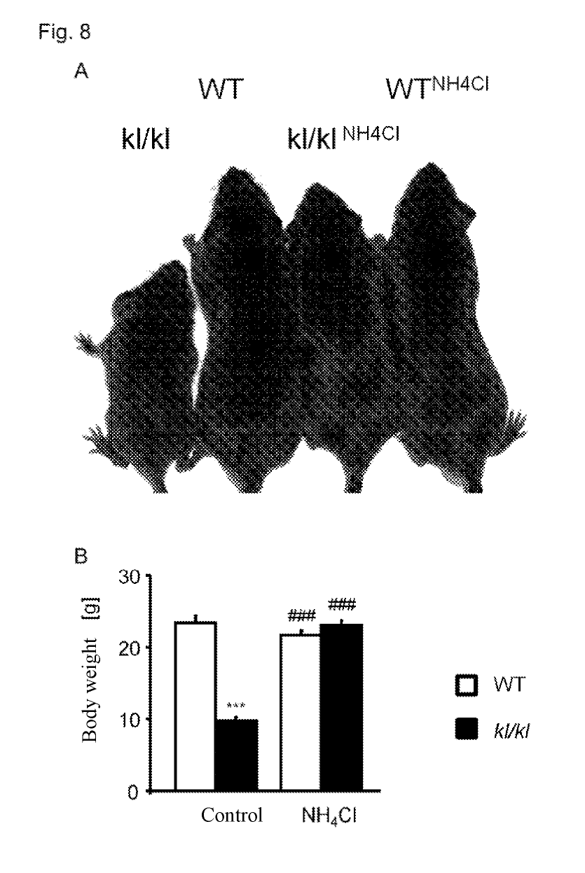

FIG. 8 shows the phenotype of klotho^{+/+} mice and klotho mice, as well as the body weights of klotho^{+/+} mice (light bars) and klotho^{hm} mice (dark bars), in each case without (control) and with NH₄Cl treatment (C), n=5-7; ***(p<0.001) indicates a significant difference to klotho^{+/+} mice; ###(p<0.001) shows a statistically significant difference to untreated klotho^{hm} mice (ANOVA).

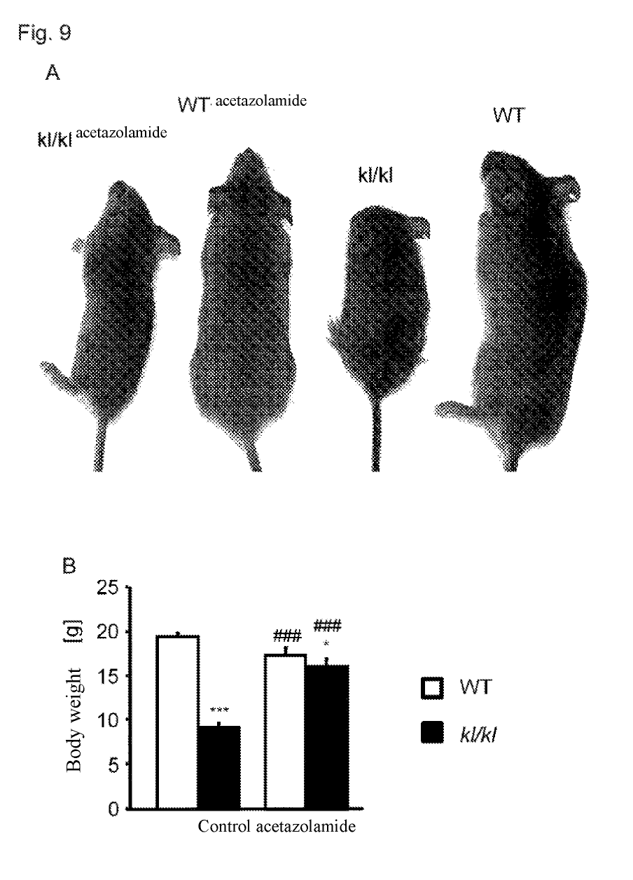

FIG. 9 shows the phenotype of klotho^{+/+} mice and klotho mice, as well as the body weights of klotho^{+/+} mice (light bars) and klotho^{hm} mice (dark bars), in each case without (control) and with acetazolamide treatment (C), n=4-6; *(p<0.05), ***(p<0.001) indicate a significant difference to klotho^{+/+} mice; ###(p<0.001) shows a statistically significant difference to untreated klotho^{hm} mice (ANOVA).

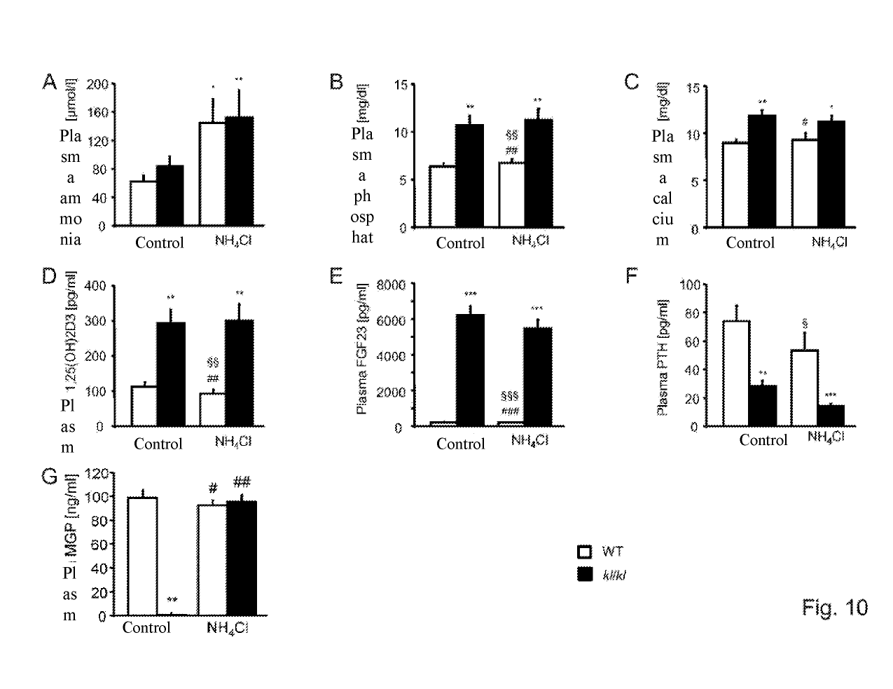

FIG. 10 shows the ammonia (A) (n=8), phosphate (B) (n=7), Ca^{+/+} (C) (n=7), 1.25(OH)₂D₃ (D) (n=6), FGF23 (E) (n=5) and parathyroid hormone (F) (n=5) and matrix gla protein (MGP) (G) (n=6) concentrations in the plasma from klotho^{+/+} mice (light bars) and klotho^{hm} mice (dark bars) without (control) and with NH₄Cl. *(p<0.05), **(p<0.01), ***(p<0.001) indicate a significant difference to klotho^{+/+} mice; #(p<0.05), ##(p<0.01), ###(p<0.001) show a statistically significant difference to untreated klotho^{hm} mice; §p<0.05), §§(p<0.01), §§§(p<0.001) show a statistically significant difference to treated klotho^{hm} mice (ANOVA).

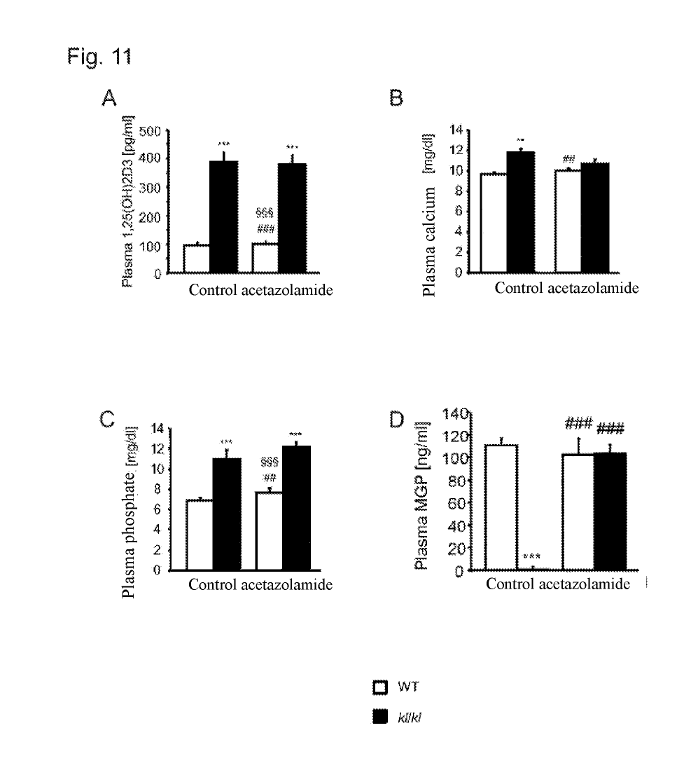

FIG. 11 shows the phosphate (A) (n=5-6), Ca^{+/+} (B) (n=5-6), 1.25(OH)₂D₃ (C) (n=5), and matrix gla protein (MGP) (D) (n=6) concentrations in the plasma from klotho^{+/+} mice (light bars) and klotho^{hm} mice (dark bars) without (control) and with acetazolamide. **(p<0.01), ***(p<0.001) show a statistically significant difference to klotho^{+/+} mice; ##(p<0.01), ###(p<0.001) show a statistically significant difference to untreated klotho^{hm} mice; §§§(p<0.001) shows a statistically significant difference to treated klotho^{hm} mice (ANOVA).

FIG. 12 shows the histology of trachea, lungs, kidneys, stomachs and vessels form klotho^{hm} mice without (untreated) or with (treated) (NH₄)₂SO₄ treatment.

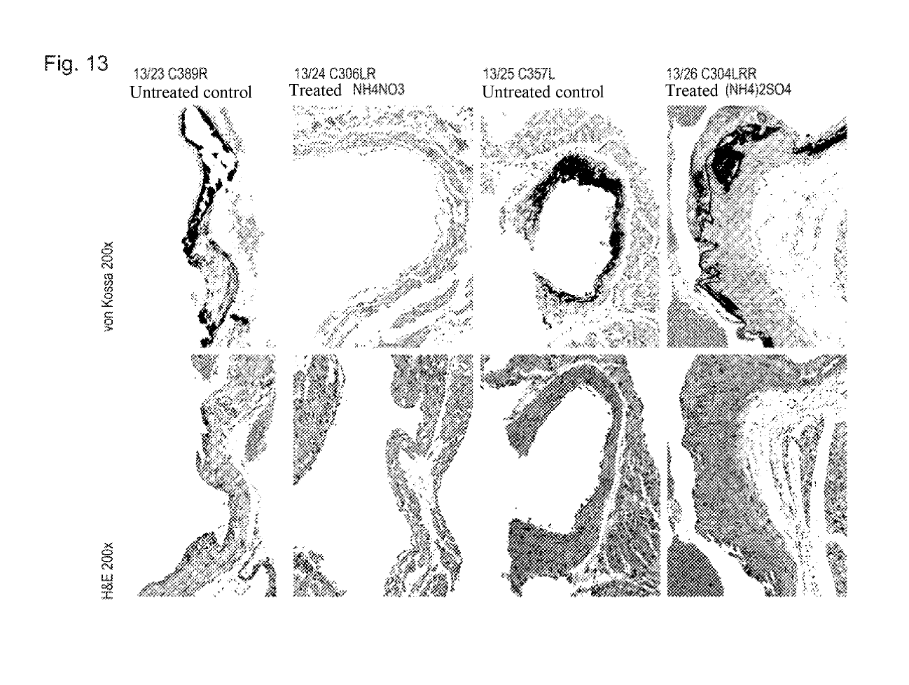

FIG. 13 shows the histology of vessels from klotho^{hm} mice without (untreated), with (treated) (NH₄)₂SO₄ or with NH₄NO₃ treatment.

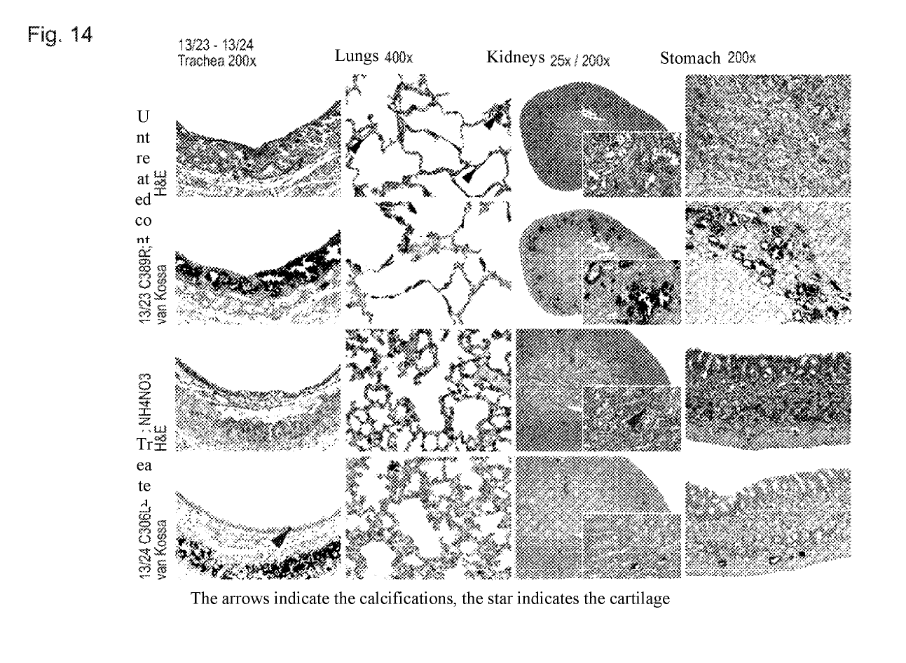

FIG. 14 shows the histology of trachea, lungs, kidneys, stomachs and vessels from klotho^{hm} mice without (untreated) and with (treated) NH₄NO₃ treatment.

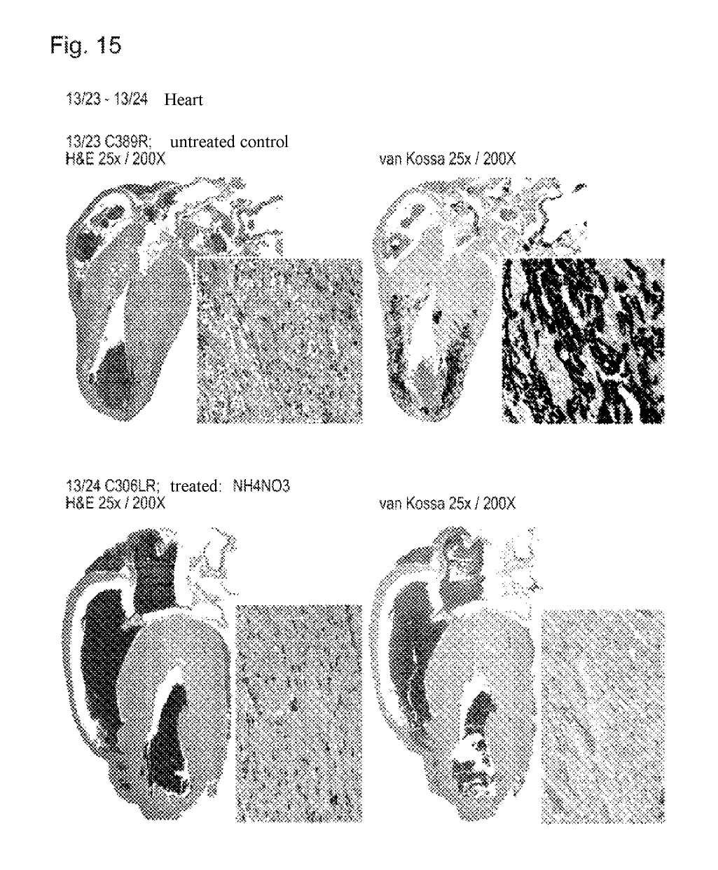

FIG. 15 shows the histology of hearts from klotho^{hm} mice without (untreated) treatment and with NH₄NO₃ treatment.

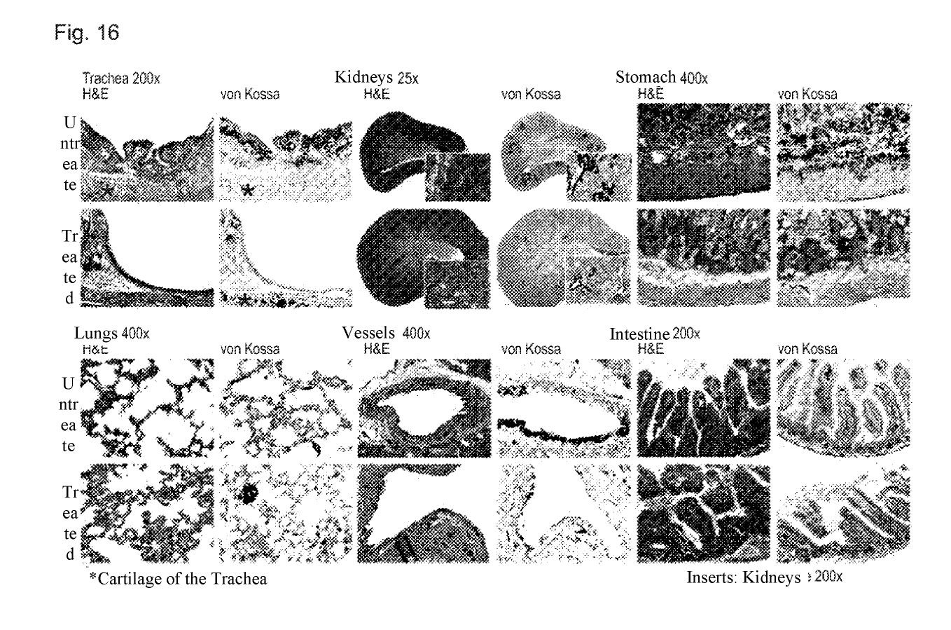

FIG. 16 shows the histology of trachea, lungs, kidneys, stomachs and vessels from klotho^{hm} mice without (untreated) and with (treated) NH₄Cl treatment.

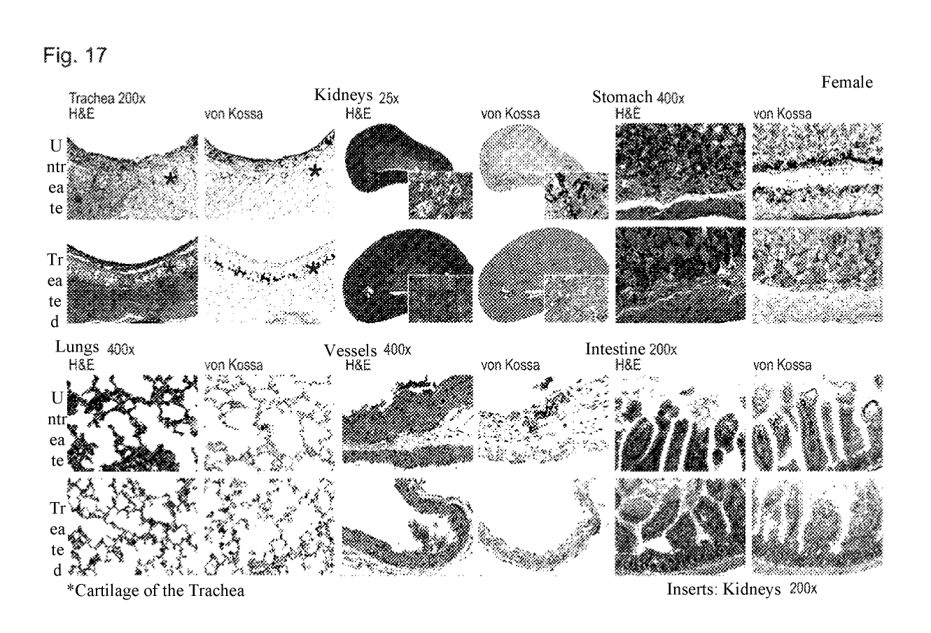

FIG. 17 shows the histology of trachea, lungs, kidneys, stomachs and vessels from klotho^{hm} mice without (untreated) and with (treated) acetazolamide treatment.

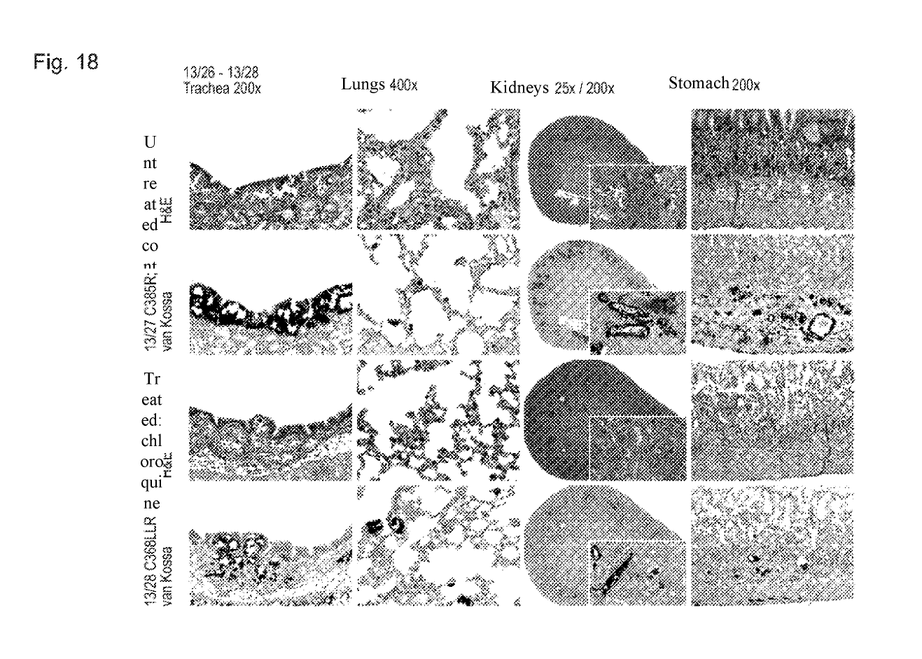

FIG. 18 shows the histology of trachea, lungs, kidneys, stomachs and vessels from klotho^{hm} mice without (untreated) and with (treated) chloroquine diphosphate treatment.

FIG. 19 shows the surviving klotho^{hm} mice without (black circle in each case) or (A) treated with (NH₄)₂SO₄ (white circle) or with NH₄NO₃ (white square) (n=9-14). (p<0.001; Wilcoxon, Log-Rang), (B) treated with NH₄Cl (white circle) (n=14-16). (p<0.001); Wilcoxon, Log-Rang), (C) treated with acetazolamide (white circle) (n=8-10). (p<0.001; Wilcoxon, Log-Rang), (D) treated with chloroquine diphosphate (white circle) (n=8-12). (p<0.05; Wilcoxon, Log-Rang).

### Materials and Methods

The following experimental studies were executed: Primary human aortic smooth muscle cells (HAoSMCs, Invitrogen) were cultivated in Waymouth's MB 752/1 medium and Ham's F12 nutrient mixture (1:1, Gibco, Life Technologies) with the addition of 10% FBS (Gibco, Life Technologies). In all experiments, confluent HAoSMCs, passages 4 to 11, were used. The cells were treated for 24 hours with 2 mM β-glycerophosphate (Sigma-Aldrich) with or without simultaneous addition of 0.5 mM ammonium salts or 100 μM chloroquine diphosphate (Sigma-Aldrich). Quantitative RT-PCR (real time polymerase chain reaction) were carried out, as described previously (Voelkl, J., Alesutan, I., Leibrock, C. B., Quintanilla-Martinez, L., Kuhn, V., Feger, M., Mia, S., Ahmed, M. S., Rosenblatt, K. P., Kuro, O., Lang, F.: Spironolactone ameliorates PIT1-dependent vascular osteoinduction in klotho-hypomorphic mice. J. Clin Invest 2013; February 1; 123(2):812-22). For this, HAoSMCs were washed and total RNA were isolated using Trifast reagent (Peqlab) according to the directions from the manufacture. For the in vivo experiments, aortas from klotho^{+/+} mice and klotho^{hm} mice, in each case with and without treatment with NH₄Cl, were removed and quick-frozen. The total RNA were likewise isolated with Trifast reagent (Peqlab) in accordance with the directions from the manufacture. In each case, 2 μg RNA of the human as well as the murine samples were used for the reverse transcription of the RNA with oligo(dT)₁₂₋₁₈ primers (Invitrogen) and SuperScripIII Reverse Transcriptase (Invitrogen). Quantitative real-time PCR were carried out with an iCycler iQ™ Real-Time PCR Detection System (Bio-Rad Laboratories) and iQ™ Sybr Green Supermix (Bio-Rad Laboratories) according to the manufacturer's instructions. The following primers were used (5′ orientation):

### Human Primers:

TN alkaline phosphatase fw:
(SEQ ID No. 1)
GGGACTGGTACTCAGACAACG;
TN alkaline phosphatase rev:
(SEQ ID No. 2)
GTAGGCGATGTCCTTACAGCC;
RUNX2 fw:
(SEQ ID No. 3)
GGAAGGGCTTGATTGACGTG;
RUNX2 rev:
(SEQ ID No. 4)
CAGAACCAAACATAGCACTGACT;
TGFB1 fw:
(SEQ ID No. 5)
CAATTCCTGGCGATACCTCAG;
TGFB1 rev:
(SEQ ID No. 6)
GCACAACTCCGGTGACATCAA;
GAPDH fw:
(SEQ ID No. 7)
GAGTCAACGGATTTGGTCG;
GAPDH rev:
(SEQ ID No. 8)
GACAAGCTTCCCGTTCTCAG;
NFAT5 fw:
(SEQ ID No. 9)
GGGTCAAACGACGAGATTGTG;
NFAT5 rev:
(SEQ ID No. 10)
GTCCGTGGTAAGCTGAGAAAG;
SOX9 fw:
(SEQ ID No. 11)
AGCGAACGCACATCAAGAC;
SOX9 rev:
(SEQ ID No. 12)
CTGTAGGCGATCTGTTGGGG;

### Murine Primer:

Nfat5 fw:
(SEQ ID No. 13)
CTGTAGGCGATCTGTTGGGG;
Nfat5 rev:
(SEQ ID No. 14)
CTGGTGCTCATGTTACTGAAGTT;
Gapdh fw:
(SEQ ID No. 15)
AGGTCGGTGTGAACGGATTTG;
Gapdh rev:
(SEQ ID No. 16)
TGTAGACCATGTAGTTGAGGTCA;

The specificity of the PCR products was checked by means of melting curve analysis and agarose gel electrophoresis. All PCRs were each carried out twice, and the multiple mRNA quantities were calculated by means of the 2^{-ΔAct} methods with GAPDH as the internal reference.

All of the animal experiments were carried out in compliance with the German Animal Protection Law, and were approved by the local authorities.

Male and female hypomorphic klotho mice (klotho^{hm}) were compared with male and female wild mice (klotho^{+/+}). The source of the mice, the breeding, and the genotyping are described in the prior art; cf. Kuro-o et al. (1997), Mutation of the mouse klotho gene leads to a syndrome resembling ageing, Nature 390: 45-51. Through repeated backcrossings (>9 generations) with animals of the 129/Sv inbreeding strain, congenic strains of the klotho mice were produced and used in this study. The mice had random access to water or an aqueous solution of (NH₄)₂SO₄ (0.14M), NH₄Cl (0.28M), NH₄NO₃ (0.28M), acetazolamide (800 mg/1) and chloroquine diphosphate (0.288 mg/ml) and were fed with a control feed (Sniff, Soest, Germany). The treatment with NH₄Cl (0.28M) or acetazolamide (800 mg/1) started with the pairing of the parental generation and was continued throughout the pregnancy, until the descendants were killed.

1.2 Blood Chemistry

For blood withdrawal, the mice were anesthetized with diethyl ether (Roth, Karlsruhe, Germany) and blood samples of 50 to 200 μl were withdrawn in capillaries containing heparin by puncturing the retro-orbital plexus. The phosphate and calcium concentrations in plasma were determined using a photometric method (FUJI FDC 3500i, Sysmex, Nordstedt, Germany). The FGF23 and PTH concentrations in plasma were determined using commercial ELISA kits (FGF23: ImmunDiagnostics, Boston, USA; PTH; Immunotopics, San Clemente, USA, MPG: Cloud-Clone Corporation, Houston, USA). The measurement of the concentration of calcitriol [1.25(OH) vitamin D₃] in plasma likewise occurred using a commercial ELISA kit (IDS, Boldon, United Kingdom). The ammonia concentration was enzymatically measured using glutamate dehydrogenase with NADPH as a cofactor. The evaluation likewise occurred with a photometric method (ADVIA 1650 analyzer, Siemens, Fernwald, Germany).

1.3 Histology

For the examination of the trachea, lunges, kidneys, heart, the stomach and the vessels, corresponding tissue was removed from male klotho^{+/+} mice (age: 8 weeks) and male klotho^{hm} mice (age: 8 weeks), without and with an aqueous solution treatment composed of (NH₄)₂SO₄ (0.14 M), NH₄Cl (0.28 M), NH₄NO₃ (0.28 M) and chloroquine (0.288 mg/ml), or in female animals without and with acetazolamide treatment (800 mg/l in drinking water) embedded in paraffin, cut into slices of 2 to 3 μm, and dyed with H&E and von Kossa; cf. Mossbrugger et al. (2007), Standardized morphological phenotyping of mouse models of human diseases within the German Mouse Clinic, Verh. Dtsch. Ges. Pathol. 91; 98-103.

1.4 Statistics

The data were presented as average±SEM, wherein n represents the number of independent experiments. All of the data were tested for significance using ANOVA or the paired or unpaired student t-tests. For the experiments regarding lifespan, SAS Jmp Version 8.0.1 (SAS Institute Inc., Cary, N.C., USA) was used. Only results having a p<0.05 were regarded as statistically significant.

a. Results

klotho is a transmembrane protein, related to the β-glucuronidase. A reduced production of this protein was observed in patients having chronic kidney failure, frequently accompanied by degenerative processes such as arterial sclerosis, osteoporosis, and skin atrophy. Mutations in these proteins were able to be connected to aging processes.

In the examined mouse model, the klotho expression was massively reduced by a defect in the klotho gene. The mice having this defect were referred to as hypomorphic mice. The deficit in klotho resulted in a syndrome that resembles human aging. The (accelerated) onset in these animals of tissue and/or vessel calcification, arterial sclerosis, pulmonary emphysema, skin atrophy, myasthenia, acquired immune deficiency syndrome, infertility, kyphosis, disrupted CaPO₄ metabolism, osteoporosis, low immunity (thymus degeneration), hearing loss and neurodegeneration, was observed. The animals having this defect furthermore have a significantly reduced life expectancy and are infertile.

The expression of TGFβ1 mRNA in human aortic smooth muscle cells (HAoSMCs) is depicted in FIG. 1. The cells were treated with 2 mM β-glycerophosphate, in order to stimulate the osteogenic signal transduction. The increased phosphate concentration led to a significant increase in the TGFβ1 mRNA expression. This increase was able to be lessened through the addition of various ammonium salts (0.5 mM) (ammonium lactate, ammonium citrate, ammonium sulfate) or even prevented (ammonium chloride, ammonium nitrate).

The expression of Runx2 mRNA in human aortic smooth muscle cells (HAoSMCs) is shown in FIG. 2. Here as well, an increase in the phosphate concentration through the addition of 2 mM β-glycerophosphate led to an increased expression of the transcription factor, which in turn could be suppressed through the addition of ammonium chloride, ammonium nitrate, and ammonium sulfate.

Furthermore, the expression of the alkaline phosphatase (ALP) in human aortic smooth muscle cells (HAoSMCs) was studied (FIG. 3). The addition of 2 mM β-glycerophosphate led in turn to increased ALP mRNA expression. FIG. 3A shows the suppression of the expression increase by means of ammonium chloride, ammonium nitrate, and ammonium sulfate. FIG. 3B shows the suppression of the ALP mRNA expression increase by means of chloroquine (100 μM).

It is shown in FIG. 4 that the expression of the transcription factor NFAT5 in aortas of klotho^{hm} mice is significantly increased in comparison with the klotho^{+/+} mice. Treatment with NH₄Cl (0.28 M) led to a normalization of the transcription level.

FIG. 5 shows the transcription level of NFAT5 and SOX9 in human aortic smooth muscle cells (HAoSMCs). Stimulation with 2 mM β-glycerophosphate increased the transcription level, while at the same time, treatment with NH₄Cl again reduced the expression.

FIG. 6 shows the transcription level of NFAT5, SOX9, Runx2 and ALP in human aortic smooth muscle cells after transfection with NFAT5. The stimulation of the cells with 2 mM β-glycerophosphate led, however, to an increase in the respective transcription level. While a simultaneous treatment with NH₄Cl allowed the expression of the respective genes of the cells transfected with empty vectors to sink back to a normal level, the expression in the cells transfected with NFAT5 remained high.

FIG. 7 shows the rise in the transcription level of NFAT5 in human aortic smooth muscle cells treated with TGFβ and 2 mM β-glycerophosphate. While a treatment of the cells with NH₄Cl was able to reverse the increase in the NFAT5 transcription triggered by the treatment with 2 mM β-glycerophosphate, with a simultaneous treatment with TGFβ, the expression remained significantly increased.

The clear growth deficit of untreated hypomorphic klotho mice (klotho^{hm}) in comparison with their wild cousins (klotho^{+/+}) is shown in FIG. 8. The growth deficit of the klotho^{hm} mice could be nearly entirely neutralized through treatment with NH₄Cl (klotho^{hm}) NH₄Cl). Wild mice displayed no growth stimulation in reaction to treatment with NH₄Cl (klotho^{+/+} NH₄Cl). As can be seen in FIG. 8, the body weight of untreated klotho^{hm} mice (control) is significantly lower than that of untreated klotho^{+/+} mice. It was possible to nearly entirely neutralize the weight deficiency in klotho^{hm} mice treated with NH₄Cl (B).

As is depicted in the following table, the pH value of the blood from untreated klotho mice was significantly lower than from untreated klotho^{+/+} mice. In klotho^{+/+} mice, the administration of NH₄Cl tends to lead to a lowering of the pH value in blood, but does not achieve statistical significance. Accordingly, a treatment of klotho^{hm} mice with NH₄Cl also does not lead to a significant increase in acidosis (Table 1).

TABLE 1
pH value in blood from wild mice (klotho^{+/+}) and hypomorphic
klotho mice (klotho^{hm}), which have received either water or an
aqueous NH₄Cl solution (15 g/l) (mathematical average ±
SEM, n = 4, *p < 0.05 indicates a statistically significant
difference to klotho^{+/+} animals that drank water).

| Mice | Drinking liquid | pH vale |
|---|---|---|
| klotho^{+/+} | water | 7.42 ± 0.03 |
| klotho^{+/+} | NH⁴Cl | 7.39 ± 0.04 |
| klotho^{hm} | water | 7.33 ± 0.02* |
| klotho^{hm} | NH₄Cl | 7.32 ± 0.3* |

The treatment of klotho^{hm} mice with acetazolamide likewise led to an increase in weight and size in the animals. As can be seen in FIG. 9, the growth deficit could not be fully compensated for by the treatment. The body weight, depicted in FIG. 9B, could be increased by acetazolamide, but the animals remained significantly smaller than klotho^{+/+} mice.

As is depicted in FIG. 10A, the treatment of the mice with ammonium chloride led to a significant increase in the ammonia concentration in plasma, both for klotho^{hm} mice as well as klotho^{+/+} mice. FIG. 10B shows the plasma phosphate level in the animals, which was significantly higher in klotho mice than in klotho^{+/+} mice. A treatment with NH₄Cl altered neither the plasma phosphate level of klotho^{+/+} mice, nor of klotho^{hm} mice. As is shown in FIG. 10C, the plasma concentrations of Ca⁺⁺ in untreated klotho^{hm} mice was significantly higher than in klotho^{+/+} mice. The NH₄Cl treatment tends to lead to a reduction in the Ca⁺⁺ concentrations in plasma from klotho^{hm} mice, but did not achieve a statistical significance. Likewise depicted in FIG. 10 are the plasma concentration of 1.25 (OH)₂D₃ (calcitriol), FGF23 and parathyroid hormone. The concentrations of 1.25 (OH)₂D₃ and FGF23 were significantly higher, the concentrations of parathyroid hormone were significantly lower in klotho^{hm} mice than in klotho^{+/+} mice. The treatment of klotho^{hm} mice with NH₄Cl did not lead to a significant change in the hormone concentrations (FIGS. 10D-F). Accordingly, the plasma concentrations of calcitriol, or 1.25 (OH)₂D₃ and of FGF23 remained significantly higher in klotho^{hm} mice than in klotho^{+/+} mice, even after treatment with NH₄Cl. Furthermore, klotho^{hm} mice have a significantly lower concentration of matrix gla protein (MGP) in plasma, which could be used as a standard for treatment with NH₄Cl (FIG. 10G).

The plasma concentrations of Ca⁺⁺, phosphate and 1.25 (OH)₂D₃ in klotho^{+/+} mice are depicted, in each case with and without treatment with acetazolamide. Neither the concentrations of 1.25 (OH)₂D₃ nor phosphate are affected by the treatment thereby. The calcium level in the treated klotho^{hm} mice also remained slightly elevated, but displayed a significant different to neither the untreated klotho^{hm} mice nor the klotho^{+/+} mice. It was also possible to fully normalize the concentration of matrix gla protein (MGP) in the animal plasma through treatment with acetazolamide.

As is shown in FIGS. 12-18, strong calcification was observed in klotho^{hm} mice that were 8 weeks old in all of the analyzed tissues, e.g. the trachea, lungs, kidneys, stomach and vessel tissues. It was possible to strongly reduce the calcification in klotho^{hm} mice through a treatment with (NH₄)₂SO₄ (FIGS. 12, 13), NH₄NO₃ (FIGS. 13, 14, 15) and NH₄Cl (FIG. 16).

FIG. 17 likewise shows histological sections of selected organs of klotho mice. The treatment of the animals with acetazolamide likewise led to a significant reduction of the calcification in the analyzed tissues.

FIG. 18 shows histological sections of selected organs from klotho^{hm} mice and klotho^{hm} mice treated with chloroquine diphosphate. The treatment of the animals with the chloroquine salt likewise led to a significant reduction of the calcification in the analyzed tissues.

As is depicted in FIG. 19, the treatment of klotho^{hm} mice with (NH₄)₂SO₄, NH₄Cl, NH₄NO₃, acetazolamide or chloroquine led to a drastic extension of the lifespans. In comparison with untreated klotho^{hm} mice, which died at an average age of 66 days, the treatment of the animals with (NH₄)₂SO₄ could extend the average life expectancy to 129 days, the treatment with NH₄Cl could extend the average life expectancy to 112 days (FIG. 19A) (n=9-14). The treatment with NH₄Cl could also significantly extend the lifetime of the animals in a further experiment. While all klotho^{hm} mice (n=16) died after 110 days, all of the mice treated with NH₄Cl survived this long (n=14) (FIG. 19A). Untreated klotho^{hm} mice (n=10) had, in contrast, an average lifetime of 78 days in this experiment. Acetazolamide also displayed a strong effect on the lifetime of the animals (FIG. 19B), even though this was not as strongly pronounced as in the treatment with NH₄Cl. The average life expectancy of the animals treated with acetazolamide was 220 days. The animals in the control group (n=10) died in this case after 90 days. At this time, all of the animals treated with acetazolamide were still alive. Furthermore, the effects of chloroquine on the lifetime of klotho^{hm} mice were also examined. The average life expectancy of the klotho^{hm} mice treated with chloroquine diphosphate rose to 90 days (FIG. 19D).

3. Conclusion

The inventors provided substances with ammonium sulfate, ammonium chloride (NH₄Cl), acetazolamide, chloroquine, ammonium nitrate, ammonium citrate, and ammonium lactate, which are suitable for preventing tissue calcification and tissue fibrosation, and delaying the onset of age-related diseases, and thus to extend the lifetime of a living being. On one hand, this was demonstrated by the effects on the expression of calcification and fibrosation indicators in the cell culture, and on the other hand, by the impressive effects of ammonium chloride and acetazolamide on an established animal model. The animals treated accordingly display significantly reduced aging syndromes, impressively illustrated by the tissue and vessel calcification, and live significantly longer than untreated animals.

1. A use of a substance selected from the group composed of: ammonium sulfate, ammonium chloride, acetazolamide, chloroquine, ammonium nitrate, ammonium citrate and ammonium lactate, for reducing tissue calcification and tissue fibrosation, as well as for delaying the onset of age-related diseases in a living being.

2. The use according to claim 1, characterized in that the age-related disease is selected from the group composed of: arterial sclerosis, pulmonary emphysema, skin atrophy, myasthenia, acquired immune deficiency syndrome, infertility, kyphosis, disrupted CaPO₄ metabolism, osteoporosis, low immunity (thymus degeneration), and neurodegeneration.

3. The use according to claim 1, characterized in that the tissue fibrosation is based on a disease that is selected from the group composed of: renal failure, cirrhosis of the liver, Crohn's disease, fibrotic pancreatitis, pulmonary fibrosis, heart failure, scarring, fibrosation in peritoneal dialysis, and Alzheimer's disease.

4. The use according to claim 1, characterized in that the substance is used as the active substance in a pharmaceutical composition.

5. The use according to claim 4, characterized in that the pharmaceutical composition is designed for an application selected from the group composed of: oral, rectal, parenteral, intraperitoneal, local, and transdermal.

6. The use according to claim 4, characterized in that the pharmaceutical composition is created in a form selected from the group composed of: powder, tablet, juice, drops, dialysis liquid, capsule, suppository, solution, injection solution, aerosol, ointment, rinse, bandage, pellet, pill, and modified release dosage form.

7. The use according to claim 1, characterized in that the substance(s) is/are used as an additive in a food product.

8. A method for producing a pharmaceutical composition for reducing tissue calcification and tissue fibrosation, for delaying the onset of age-related diseases of a living being, having the following steps:
1. provision of an active substance, and
2. formulation of the active substance in a pharmaceutically acceptable carrier for obtaining the pharmaceutical composition,
characterized in that the active substance is selected from the group composed of: ammonium sulfate, ammonium chloride, acetazolamide, chloroquine, ammonium nitrate, ammonium citrate, and ammonium lactate.

9. A method for producing a food product for reducing tissue calcification and tissue fibrosation, for delaying the onset of age-related diseases of a living being, having the following steps:
1. provision of an active substance, and
2. formulation of the active substance in a pharmaceutically acceptable carrier for obtaining the food product,
characterized in that the active substance is selected from the group composed of: ammonium sulfate, ammonium chloride, acetazolamide, chloroquine, ammonium nitrate, ammonium citrate, and ammonium lactate.

10. A method for reducing tissue calcification and tissue fibrosation, for delaying the onset of numerous age-related diseases in a living being, which comprises the administration of a substance to the living being, which is selected from the group composed of: ammonium sulfate, ammonium chloride, acetazolamide, ammonium nitrate, ammonium citrate, and ammonium lactate.


SEQUENCE LISTING (17 sequences)

SEQ ID NO: 1 - DNA, 21 bp, Artificial Sequence
  Description of Artificial Sequence Synthetic primer
gggactggtactcagacaacg

SEQ ID NO: 2 - DNA, 21 bp, Artificial Sequence
  Description of Artificial Sequence Synthetic primer
gtaggcgatgtccttacagcc

SEQ ID NO: 3 - DNA, 20 bp, Artificial Sequence
  Description of Artificial Sequence Synthetic primer
ggaagggcttgattgacgtg

SEQ ID NO: 4 - DNA, 23 bp, Artificial Sequence
  Description of Artificial Sequence Synthetic primer
cagaaccaaacatagcactgact

SEQ ID NO: 5 - DNA, 21 bp, Artificial Sequence
  Description of Artificial Sequence Synthetic primer
caattcctggcgatacctcag

SEQ ID NO: 6 - DNA, 21 bp, Artificial Sequence
  Description of Artificial Sequence Synthetic primer
gcacaactccggtgacatcaa

SEQ ID NO: 7 - DNA, 20 bp, Artificial Sequence
  Description of Artificial Sequence Synthetic primer
gagtcaacggatttggtcgt

SEQ ID NO: 8 - DNA, 20 bp, Artificial Sequence
  Description of Artificial Sequence Synthetic primer
gacaagcttcccgttctcag

SEQ ID NO: 9 - DNA, 21 bp, Artificial Sequence
  Description of Artificial Sequence Synthetic primer
gggtcaaacgacgagattgtg

SEQ ID NO: 10 - DNA, 21 bp, Artificial Sequence
  Description of Artificial Sequence Synthetic primer
gtccgtggtaagctgagaaag

SEQ ID NO: 11 - DNA, 19 bp, Artificial Sequence
  Description of Artificial Sequence Synthetic primer
agcgaacgcacatcaagac

SEQ ID NO: 12 - DNA, 20 bp, Artificial Sequence
  Description of Artificial Sequence Synthetic primer
ctgtaggcgatctgttgggg

SEQ ID NO: 13 - DNA, 20 bp, Artificial Sequence
  Description of Artificial Sequence Synthetic primer
ctgtaggcgatctgttgggg

SEQ ID NO: 14 - DNA, 23 bp, Artificial Sequence
  Description of Artificial Sequence Synthetic primer
ctggtgctcatgttactgaagtt

SEQ ID NO: 15 - DNA, 21 bp, Artificial Sequence
  Description of Artificial Sequence Synthetic primer
aggtcggtgtgaacggatttg

SEQ ID NO: 16 - DNA, 23 bp, Artificial Sequence
  Description of Artificial Sequence Synthetic primer
tgtagaccatgtagttgaggtca

SEQ ID NO: 17 - DNA, 18 bp, Artificial Sequence
  Description of Artificial Sequence Synthetic primer
tttttttttttttttttt